(19) United States
(12) Patent Application Publication
(10) Pub. No.: US 2009/0247841 A1
(43) Pub. Date: Oct. 1, 2009

(54) LANCET WITH CAPILLARY CHANNEL

(76) Inventors: Gerhard Werner, Weinheim (DE); Hans-Peter Haar, Wiesloch (DE)

Correspondence Address: BOSE MCKINNEY & EVANS LLP, 111 Monument Circle, Suite 2700, INDIANAPOLIS, IN, 46204, US

(21) Appl. No.: 12/422,073

(22) Filed: Apr. 10, 2009

Related U.S. Application Data
(63) Continuation of application No. PCT/EP2007/008686, filed on Oct. 6, 2007.

(30) Foreign Application Priority Data
Oct. 14, 2006 (EP) EP 06021579.5

(51) Int. Cl. A61B 5/157; A61B 5/151

(52) U.S. Cl. 600/310; 600/583

(57) ABSTRACT

The invention relates to a lancet having a lancet body with a lancet tip for creating a puncture wound and a capillary channel to convey body fluids out of the puncture wound in a direction of conveyance. The capillary channel is formed by a slot which runs through the entire lancet body perpendicular to the direction of conveyance, and a test area for optically inspecting a body fluid sample which is conveyed by the capillary channel. The test area is constructed as a section of the slot and has a transparent component with a moistening surface for moistening with a body fluid sample for inspection. The invention further relates to a pricking system comprising such a lancet and a pricking device for creating a puncture wound.

### RELATED APPLICATIONS

This application is a continuation application of International Application PCT/EP2007/008686, filed Oct. 6, 2007, which claims priority to EP 06021579.5, filed Oct. 14, 2006, which are hereby incorporated by reference in their entirety.

### BACKGROUND

The invention relates to a lancet with a lancet body that is provided with a lancet tip to generate a puncture wound and a capillary channel for the conveyance of body fluids from a puncture wound in a conveying direction. Such a lancet is generally known, for example, from U.S. Publication No. 2003/0171699 as well as from WO 2005/084530.

Within the scope of the present application, a capillary channel shall be understood a channel that is dimensioned in such a manner that capillary forces convey a body fluid in the channel.

Lancets with capillary channels for the withdrawal of body fluids are used, e.g., by diabetics who must monitor their blood sugar level several times a day by generating a small puncture wound in a body part, usually in a finger, to obtain a body fluid sample that, in order to determine its glucose content, can be analyzed by means of a measuring device. Lancets with capillary channels have the advantage that generating the puncture wound and withdrawing a body fluid can be affected in a single step, which is of great importance especially in the case of patients who, because of age or ailments, are of diminished agility. In particular, with automatic sampling by means of the capillary channel it is possible to reduce the risk of contamination or of an erroneous test result because of improper handling of a sample.

A disadvantage of the known lancets with capillary channels is, however, their high manufacturing costs compared to the customary lancets.

### SUMMARY

The present invention provides a lancet with a capillary channel for the conveyance of body fluids from a puncture wound generated by the lancet which can be manufactured at a low cost.

While the capillary channel of the lancets known in the art is configured as a groove that causes a high manufacturing cost, a slot according to the invention can be made, especially in a steel lancet body, at a considerably lower cost, be it, e.g., by laser beam cutting, chemical etching or punching. Surprisingly, an open slot running vertically to the conveyance direction through the lancet body generates a fully sufficient, to some extent even improved, conveyance effect to obtain a body fluid sample from a puncture wound generated by the lancet.

A lancet with a test area for the optical examination of a body fluid sample conveyed by means of the capillary channel offers therefore the same advantages as lancets with capillary channels known from prior art but at considerably lower manufacturing costs. In particular, the test area can be easily and at low cost configured as a section of the slot, so that by means of a lancet according to the invention it is possible to facilitate a cost-effective testing of a body fluid sample.

The test area of a lancet comprises a transparent component provided with a wetting surface to be wetted by a body fluid sample to be tested. By way of example, the test area can be configured as a cuvette, the transparent component being a wall of the cuvette whose inside constitutes the wetting surface. It is especially advantageous to use a plastic sheet for the transparent component and to form two parallel cuvette walls by covering the slot with a plastic sheet. In such a manner it is possible to facilitate in a cost-effective manner an examination of a body fluid sample by optical transmission.

As an alternative to the examination of the body fluid sample in transmission, the transparent component can be provided with skewed lateral surfaces that can be used for the coupling and decoupling of examination light which, by means of total internal reflection, is guided along the wetting surface, thereby interacting with a sample wetting the wetting surface.

In particular, with a lancet according to these teachings, it is possible to examine a body fluid sample in a reagent-free manner. The test areas of customary lancets usually contain sensitive reagents such as, e.g., enzymes for a photometric examination, but this could hinder sterilization. A lancet according to these teachings can be provided with a reagent-free test area and therefore can be sterilized in a much simpler manner, for example, by heat and/or radiation, especially electron beams.

It is preferable that the lateral walls of the slot be coated with a hydrophilic substance in order to increase the capillary effect. In an enzyme-free test area, as provided in a lancet disclosed herein, the great freedom in selecting hydrophilic substances is advantageous because no interaction with a chemical test reaction is to be feared.

A further feature that can also be of significance by itself entails a lancet with a test area for the optical examination of a body fluid sample, wherein the test area comprises a wetting surface to be wetted with the body fluid sample, characterized in that the wetting surface is configured as the front side of a transparent component of which a first side face couples incoming light and a second side face decouples outgoing light. The first and second side faces can be configured obliquely oriented to the front side, especially at an angle of 45° or, e.g., as surface area of quarter cylinders.

Light entering on the first side face of the transparent component interacts with a body fluid sample wetting a front side of the transparent component. By means of total reflection the light is directed towards the second side face, where it is coupled out so that it can be conveyed to a detector.

The advantage of a lancet disclosed herein with such a wetting surface is especially that, because of the total reflection, only a thin layer of the body fluid sample on the front side interacts with light and that, consequently, a very small sample volume suffices to measure a concentration of an analyte such as, e.g., glucose concentration. For a reagent-free determination of an analyte concentration it is possible to use, e.g., the absorption characteristics of the analyte in the mid infrared spectral range. Details of the reagent-free determination of an analyte concentration by means of infrared spectroscopy are disclosed, e.g., in E. Diessel et al., Applied Spectroscopy 2004, 442 to 450, which in this respect is incorporated by reference in the present application. Of course, the material of the transparent component must be chosen according to the spectral range of the light used for the optical examination of the body fluid, so that the transparent component is sufficiently transparent for this light. Appropriate materials that are sufficiently transparent in the mid infrared spectral range, in the near infrared spectral range and/or the visible spectral range are, in particular, glass and plastic and, especially, polyethylene and polycarbonate.

Further details and features of the invention will become clear from the following description of illustrative embodiments. The respective features can be embodied either singly or in several combinations with one another, though the invention is not limited to the illustrative embodiments.

### BRIEF DESCRIPTION OF THE DRAWINGS

The above-mentioned aspects of the present invention and the manner of obtaining them will become more apparent and the invention itself will be better understood by reference to the following description of the embodiments of the invention, taken in conjunction with the accompanying drawings, wherein:

FIG. 1 is an exploded perspective view of a lancet according to one embodiment of the invention;

FIG. 2 is a perspective view of the embodiment illustrated in FIG. 1;

FIG. 3 is a top view of the embodiment illustrated in FIG. 1;

FIG. 4 is a front view of the embodiment illustrated in FIG. 1;

FIG. 5 is a rear view of the embodiment illustrated in FIG. 1;

FIG. 6 is a longitudinal sectional view of the embodiment illustrated in FIG. 1 taken along the capillary channel;

FIG. 7 shows a detailed view of FIG. 6;

FIG. 8 is a perspective view of the lancet body with the lancet tip of the embodiment illustrated in FIG. 1;

FIG. 9 is a top view of the lancet body illustrated in FIG. 6;

FIG. 10 is a front view of the lancet body of FIG. 8;

FIG. 11 is a rear view of the lancet body of FIG. 8;

FIG. 12 is a longitudinal sectional view taken along the capillary channel of the lancet body illustrated in FIG. 8;

FIG. 13 is a plan view of another embodiment of a lancet according to the invention;

FIG. 14 is a perspective view of an embodiment of a measuring device for the determination of a concentration of a body fluid sample to be analyzed in the test area of the lancet illustrated in FIG. 13;

FIG. 15 is a sectional view of the measuring device illustrated in FIG. 14;

FIG. 16 is an exploded perspective view of another embodiment of a lancet according to the invention;

FIG. 17 is a perspective view of a diagonal view of the embodiment illustrated in FIG. 16;

FIG. 18 is a top view of the embodiment illustrated in FIG. 16;

FIG. 19 is a front view of the embodiment illustrated in FIG. 16;

FIG. 20 is a rear view of the embodiment illustrated in FIG. 16;

FIG. 21 is a longitudinal sectional view taken along the capillary channel of the embodiment illustrated in FIG. 16;

FIG. 22 is a detailed view of FIG. 21;

FIG. 23 is a detailed view of another embodiment of the optical features of the capillary channel of FIG. 21; and

FIG. 24 is a diagrammatic view of an embodiment of a pricking system comprising a lancet and a pricking device.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

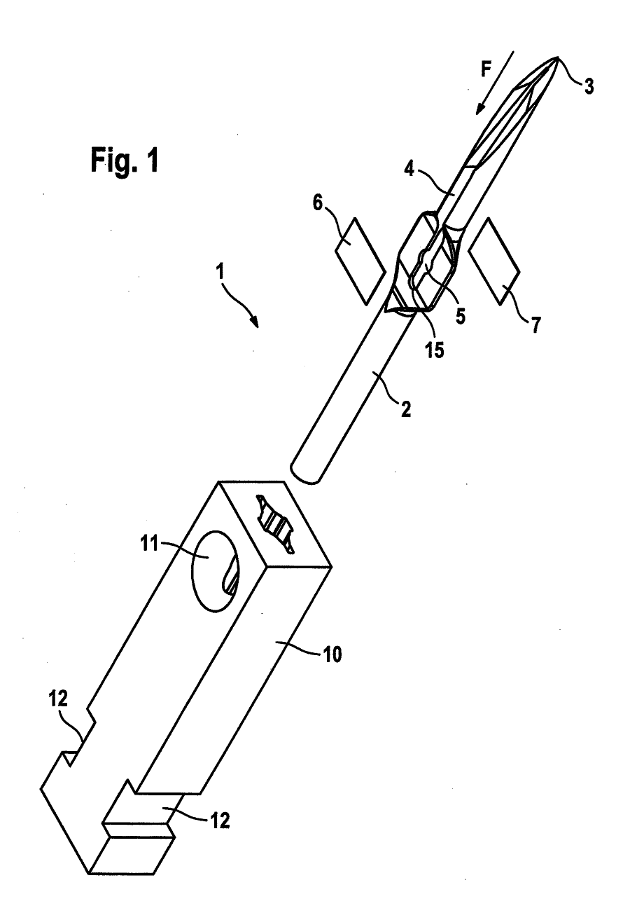

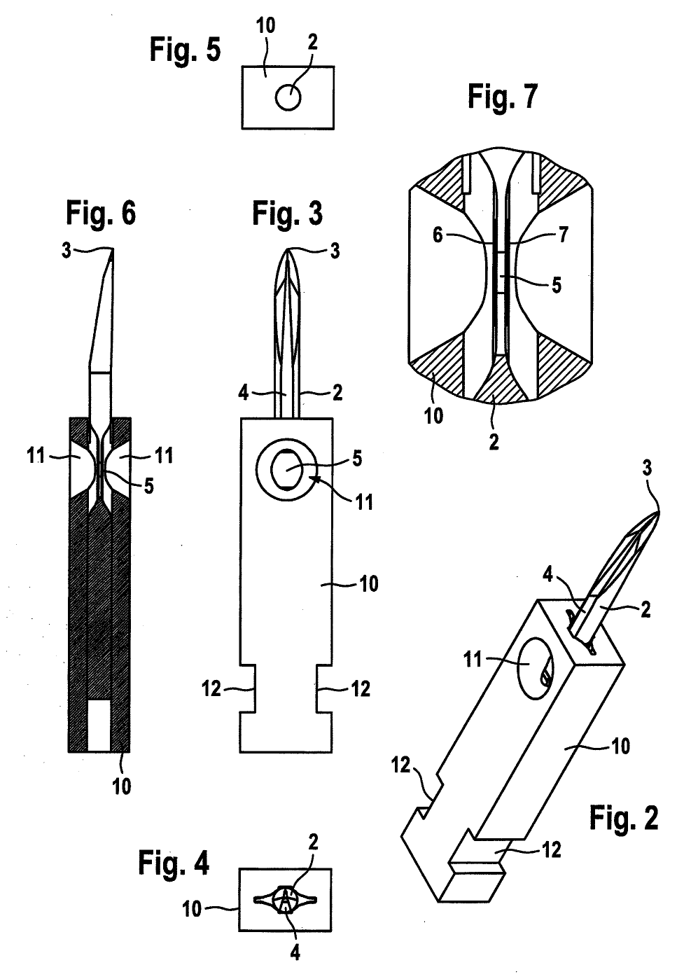

The lancet 1 depicted in FIGS. 1 to 3 comprises a steel lancet body 2 with a lancet tip 3 and a capillary channel 4 starting at the lancet tip 3 for the conveyance of body fluid in a conveyance direction F originating from a puncture wound generated by the lancet. The conveyance direction F is naturally given by the direction of the capillary channel 4. In the illustrated embodiment, the capillary channel 4 runs to an enzyme-free test area 5 for the optical examination of a body fluid sample conveyed by means of the capillary channel 4. The test area is covered with a plastic sheet 6, 7 made out of, e.g., polyethylene. The lancet body 2 is of flat configuration in a section surrounding the test area 5. Although the lancet body in the illustrated embodiment is cylindrical, it can be completely flat and, e.g., be punched out of sheet steel or cut out by laser cutting.

The test area 5 in the depicted embodiment is configured between two opposite walls made of plastic sheet 6, 7. The walls are typically arranged parallel to each other. In such a manner, a cuvette in which a body fluid can be optically examined in transmission can be configured by simple means.

In order to facilitate filling of the test area 5 with body fluid, it is advantageous to provide the test area 5 with a vent hole 15 for venting gases from the test area 5. Such a vent hole can be configured such that, adjacent to the test area 5 on the side opposite of the tip 3, there is a section of the slot that is open at least on one side, i.e., not covered by plastic film.

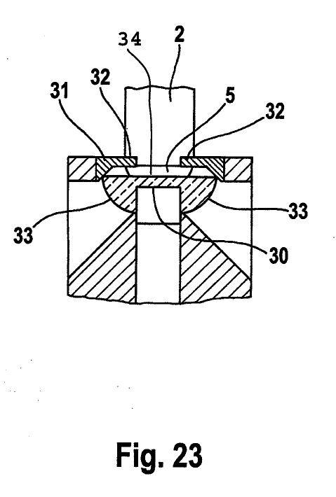

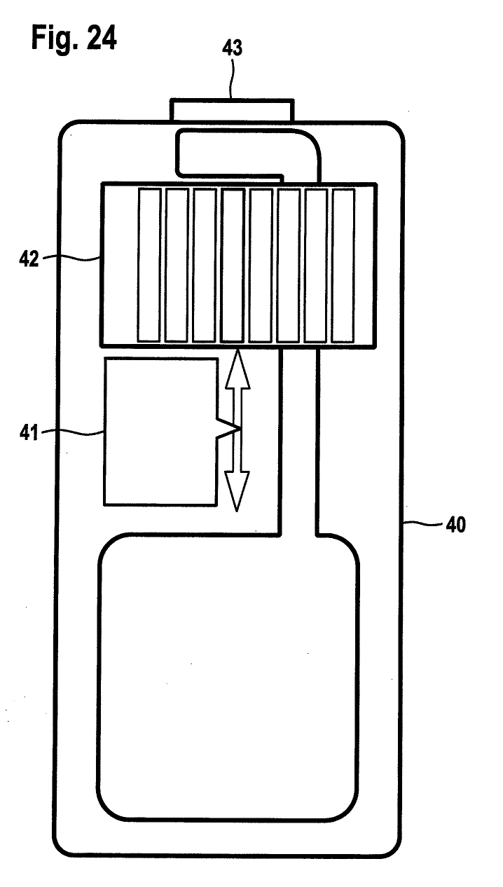

The lancet body 2 is inserted in a plastic holder 10 that is provided with opposite openings 11 for an optical examination of body fluids in the test area 5. Furthermore, the holder 10 is provided with coupling elements 12 which in the illustrated embodiment are configured as recesses by means of which the lancet 1 can be coupled to a lancet drive of a pricking device diagrammatically illustrated in FIG. 23.

FIG. 4 shows the lancet described by FIGS. 1 to 3 in a front view, i.e., with a view to the tip 3. Conversely, FIG. 5 shows a rear view of the lancet 1.

The capillary channel 4 of the lancet 1 is constituted by a slot that extends perpendicular to the conveyance direction F through the entire lancet body 2. The lateral walls of the slot 4 are covered with a hydrophilic substance such as, e.g., sorbate, in order to increase the capillary effect.

FIG. 6 shows a longitudinal section through the lancet 1 along the capillary channel 4. Therein, the test area 5 is configured as a section of the slot forming the capillary channel 4. Therefore, the slot 4 is wider in the section constituting the test area 5 than at the tip 3. Although in the illustrated embodiment the capillary channel 4 starts at the lancet tip 3, a small area of the lancet tip 3 itself is not slotted in order to facilitate a greater mechanical stability and sharpness for a painless pricking process. However, it is also possible to have the slot 4 run through the front most area of the lancet tip 3 and to have it exit in the front of the lancet tip.

FIG. 7 shows a detailed view of FIG. 6. It illustrates the test area 5 with the flat section of the lancet body 2 that surrounds it as well as the transparent plastic sheet 6 and 7 covering the test area 5.

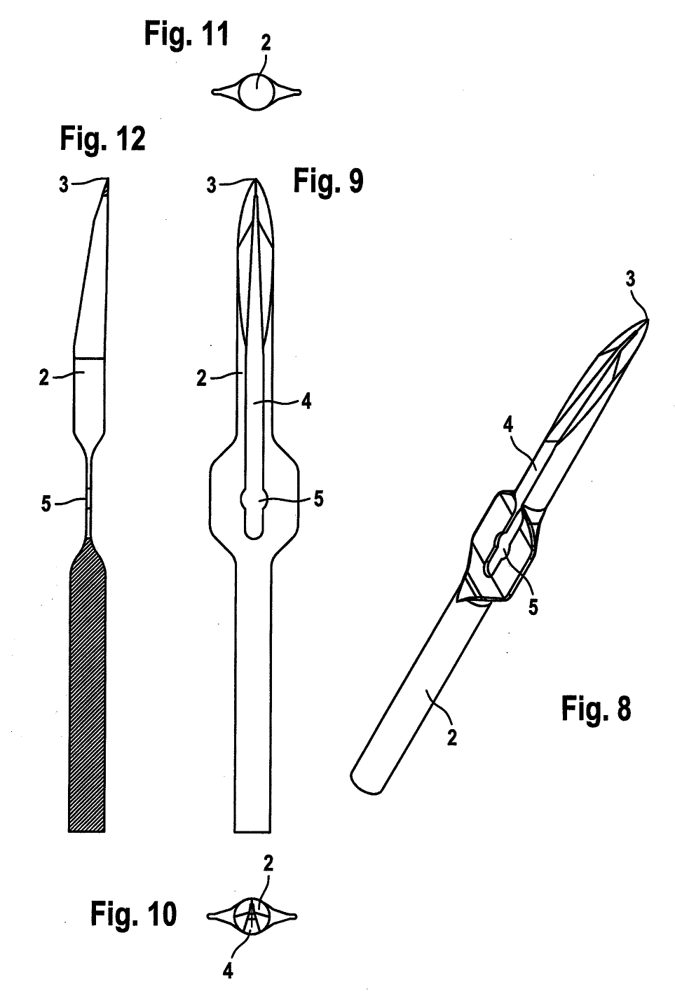

FIGS. 8 to 12 show the lancet body 2 with the lancet tip 3. FIG. 8 shows a perspective view of the lancet body 2, FIG. 9 a top view, FIG. 10 a front view towards the lancet tip, FIG. 11 a rear view, and FIG. 12 a longitudinal section along the capillary channel 4. From these figures, it can be seen that the lancet body 2 is configured flat in a section surrounding the test area 5 and cylindrical in the sections ahead and behind said test area. The cylindrical front section of the lancet body is chamfered in order to form a sharp tip 3 facilitating painless pricking into body tissue.

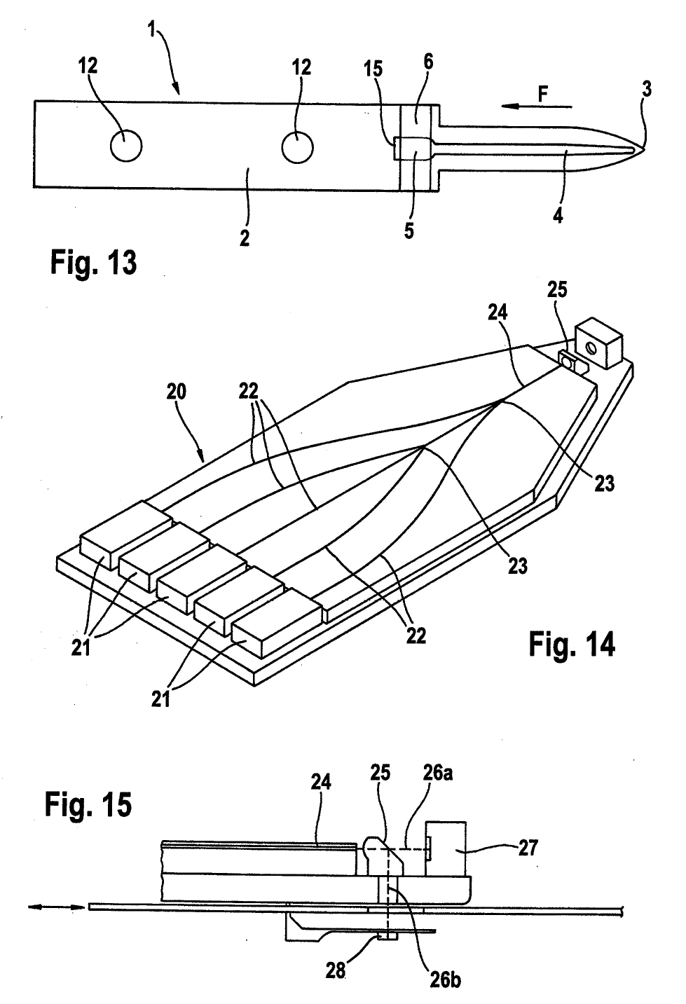

FIG. 13 shows another embodiment of a lancet 1. This lancet 1 also comprises a steel lancet body 2 with a lancet tip 3 for generating a puncture wound and a capillary channel 4 for conveying body fluid from a puncture wound in a conveyance direction F. As in the previous embodiment, the capillary channel 4 is also formed by a slot that extends perpendicular to the conveyance direction F through the entire lancet body 2. The embodiment depicted in FIGS. 11-13 is a flat lancet as the lancet body 2 is cut out of a metal strip which by, e.g., punching out or laser cutting is possible in a cost-effective manner. The capillary channel 4, including the test area 5, as well as recesses such as coupling element 12 for coupling of the lancet 1 to a drive of a pricking device can also be manufactured in a cost-effective manner by cutting the lancet body 2 from a metal strip. The slot 4 constituting the capillary channel runs in a straight line from the area of the lancet tip 3 to a widened section that forms the test area 5. The test area 5 is configured between two transparent plastic films 6, so that cuvette walls are provided for a transmission-optical examination of a body fluid sample in the test area 5. The test area 5 is provided with a vent hole 15 for the exhaust of gas at the entering of body fluid into the test area 5, for an easier filling of the test area 5 with body fluid.

Further cost savings can be obtained if the plastic sheet 6 that covers the test area 5 is part of a larger sheet that covers the entire lancet 1 for a sterile packaging. This larger sheet is glued to the lancet body 2 in the strip-shaped area depicted in FIG. 13 so that the cuvette walls remain intact when the sterile packaging is ripped open in the area of the lancet tip 3.

The test area 5 can contain analytical reagents for the photometric analysis of an analyte such as, e.g., glucose. Appropriate analytical reagents on test strips have been in use for a long time for photometric glucose determination and therefore do not require a more detailed explanation. In the presence of glucose, such analytical reagents cause discoloration that can be photometrically used for determining concentration. In the embodiment shown in FIG. 13, the test area 5 is, however, free of analytical reagents. The determination of an analyte concentration is done by a transmission measuring at several, e.g., five wavelengths in which the analyte shows a characteristic absorption behavior. By way of example, in the wavelength range of 5 μm to 15 μm the absorption spectrum of glucose contains some characteristic absorption regions that could be used for the identification and concentration determination.

FIG. 14 shows a diagrammatic view of a measuring device 20 by means of which an analyte concentration in a body fluid can be determined in the test area 5 of the embodiment shown in FIG. 13. The measuring device 20 comprises several radiation sources 21 each of which can generate a narrow-band light of a wavelength in which the analyte shows a characteristic absorption behavior. In the illustrated embodiment, the radiation sources 21 are MIR-LED, preferably cascade semiconductor lasers that can generate a laser radiation in the mid infrared spectral range. The laser light generated by the radiation sources 21 of the mid infrared spectral region is directed by means of waveguides 22 and funneled to a single waveguide 24 by means of beam-combiners 23. The waveguide 24 conveys the infra red radiation of the radiation sources 21 to a beam splitter 25 that generates two partial beams 26a and 26b that are shown in the illustrated cross-sectional view of FIG. 15 shown in FIG. 14. One partial beam 26a is conveyed as reference beam to a detector 27. The other partial beam 26b is conveyed through the test area 5 and a therein contained fluid sample to a spectral-sensitive detector 28. By comparing the signals of both detectors 27 and 28 for the individual wavelengths of the radiation source 21 it is possible to determine which fraction of the pertinent wavelength was absorbed. From this fraction it is possible to determine the analyte concentration of the body fluid sample contained in the test area 5.

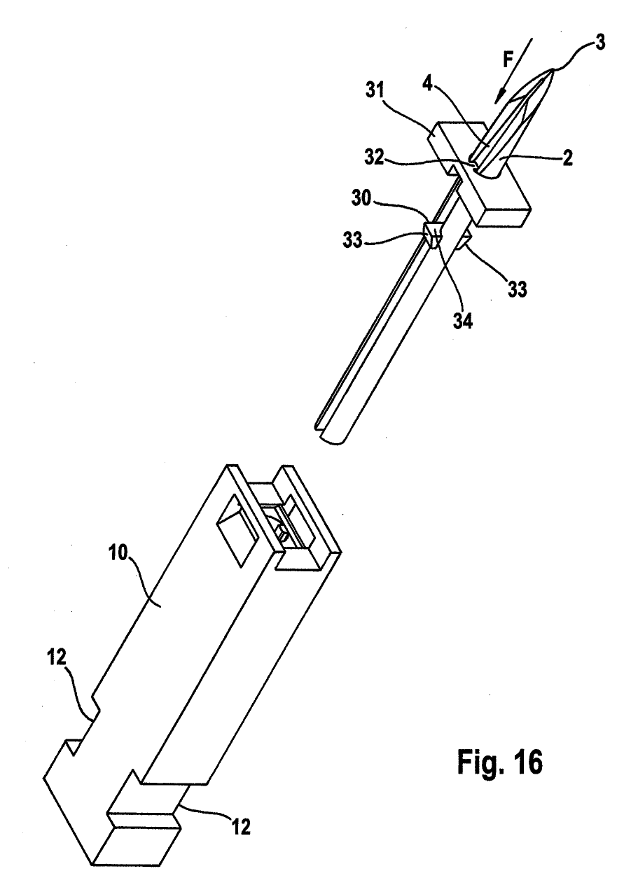

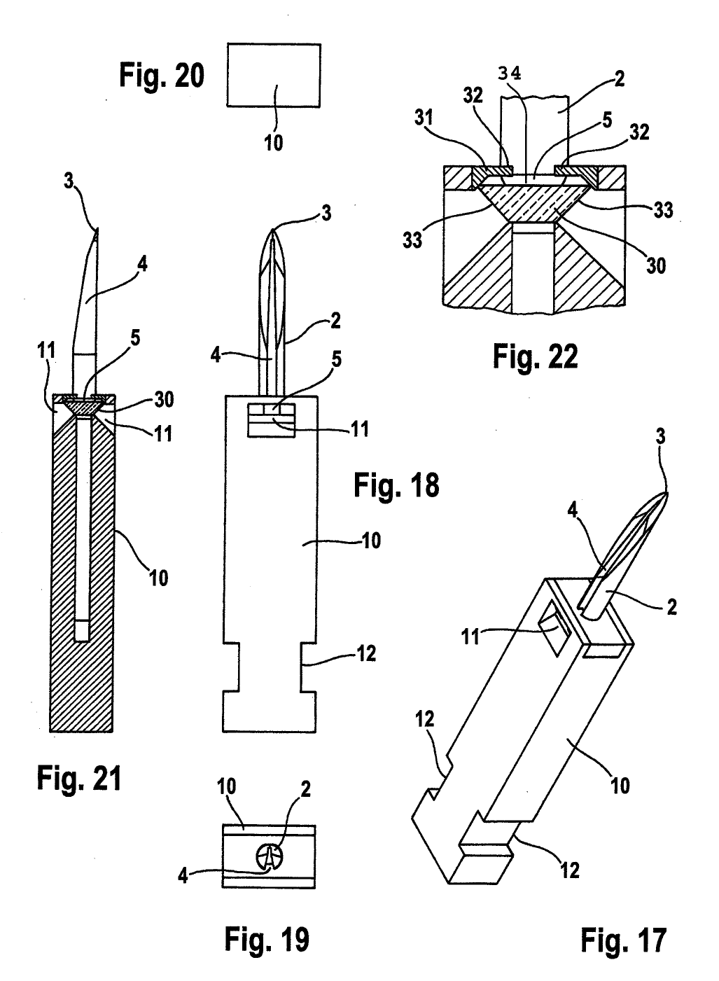

FIGS. 16 to 18 show another embodiment of a lancet with a capillary channel 4 starting at the lancet tip 3. This embodiment differs from the embodiment shown in FIG. 1 to 3 primarily by the configuration of the test area 5. The test area 5 is depicted in FIG. 21 that shows a sectional view through the lancet 1 along the capillary channel 4 and it can be seen especially in FIG. 22 that shows a detail view of FIG. 21.

The test area 5 is formed by a section of the slot constituting the capillary channel 4, which extends perpendicular to the conveyance direction F through the entire lancet body 2. The test area 5 is delimited on one side by the front side of the transparent component 30 inserted through the slot 4 and on the other side by a cap 31 that is slipped on the lancet body 2. The cap 31 is provided with two opposite projections 32 that protrude somewhat into the capillary channel 4 so that between the two projections 32 remains a clearance through which a body fluid sample can reach the test area 5.

The transparent component 30 of the embodiment shown in FIG. 22 is provided with two opposite lateral faces 33 that are chamfered at a 45° angle, measured from the front side 34 facing the test area 5. The front side 34 of the carrier 30 constitutes a wetting surface which, for the examining of a body fluid sample, is wetted with the body fluid sample. If light for examining a sample in the test area 5 is beamed through the opening 11 of the holder 10 to the first side face 33 underneath of it, this light is repeatedly fully reflected in a limiting area between the front side 34 of the transparent component 30 and a body fluid sample wetting it until it emerges on the second side face 33 from where it can be conveyed to a detector. This measuring principle is called “attenuated total reflection” and is explained, e.g., in Applied Optics, Volume 42, pages 745 to 749, by Y. Kim et al. With respect to details for the carrying out of ATR measurements, this publication is incorporated by reference in this application.

As shown in FIG. 23, the opposite side faces 33 can also be configured as surface areas of quarter cylinders. The embodiment shown in FIG. 23 has the advantage of an especially robust single-beam and emission geometry.

The transparent component 30 is preferably formed from a synthetic material, especially polyethylene or polycarbonate. The first lateral face 33 for coupling incoming light and the second lateral face 33 for decoupling outgoing light are in an oblique direction towards the front side 30 and opposite to the test area 5. Therefore, the two lateral faces 33 are not wetted during the examination of a body fluid sample.

FIG. 24 shows diagrammatically a pricking device 40 which, in conjunction with the described lancet 1, constitutes a pricking system for creating a puncture wound. The pricking device 40 is provided with a press-on element by means of which said pricking device can be pressed against a body part of a user in order to generate a puncture wound. Until being used, the lancets 1 in the pricking device 40 are housed in a magazine 42. The depicted pricking device 40 is provided with a diagrammatically illustrated lancet drive 41 for moving a lancet 1 inserted in the pricking device 40 for a pricking movement, a measuring device 20 comprising a light source and a detector for the optical examination of a body fluid sample in the test area 5 of a lancet 1 inserted in the pricking device 40.

The pricking device 40 and the lancet 1 are configured such that for optical examination of a body fluid sample in the test area 5 of a lancet 1 inserted into the pricking device 40, light emitted by the light source impinges on the side face 33, depicted in FIG. 22, of a transparent component 30 delimiting the test area of the lancet and by means of repeated total refection is conveyed to the detector through an interface region between the wetting surface 34 and a body fluid sample wetting the wetting surface.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A lancet, comprising:
a lancet body having a lancet tip for creating a puncture wound and a capillary channel to convey body fluid from a puncture wound in a conveyance direction, the capillary channel defining a slot that extends perpendicular to the conveyance direction and extends through the lancet body; and
a test area for optical examination of a body fluid sample conveyed by means of the capillary channel, the test area being configured as a section of the slot and comprising a transparent component with a wetting surface for wetting a body fluid sample to be examined.

2. The lancet of claim 1, wherein the transparent component comprises a cuvette having an inside wall that comprises the wetting surface.

3. The lancet of claim 2, wherein the transparent component comprises a plastic sheet.

4. The lancet of claim 3, wherein the cuvette comprises two opposite walls that are formed from plastic sheets that cover the slot.

5. The lancet of claim 1, wherein the transparent component has a front side comprising the wetting surface, a first lateral face for coupling incoming light, and a second lateral face for decoupling outgoing light.

6. The lancet of claim 5, wherein the first and second lateral faces are positioned obliquely relative to the front side.

7. The lancet of claim 6, wherein the first and second lateral faces are positioned at an angle of about 45° to the front side.

8. The lancet of claim 6, wherein both the first lateral face and the second lateral face are configured as the surface area of a quarter cylinder.

9. The lancet of claim 3, wherein the slot is wider in the test area than at the tip.

10. The lancet of claim 2, wherein the test area comprises a vent hole which exhausts gas from the test area.

11. The lancet of claim 1, wherein the test area is enzyme-free.

12. The lancet of claim 1, wherein the slot forming the capillary channel comprises at least at one section with a lateral face coated with a hydrophilic substance.

13. A pricking system for creating a puncture wound, comprising:
a lancet, the lancet comprising a lancet body having a lancet tip for creating a puncture wound and a capillary channel to convey body fluid from a puncture wound in a conveyance direction, the capillary channel defining a slot that extends perpendicular to the conveyance direction through the lancet body, the lancet body including a test area for optical examination of a body fluid sample conveyed by the capillary channel;
a lancet drive for driving a puncturing movement of the lancet inserted in the pricking system;
a light source and a detector for the optical examination of a body fluid sample in the test area; and
a transparent component positioned in the test area, the transparent component having a wetting surface at a front side thereof, a first lateral face for coupling incoming light and a second lateral face for decoupling outgoing light, the first lateral face and the second lateral face being positioned obliquely to the front side, wherein during use of the lancet in the pricking system, light emitted from the light source impinges upon the oblique face of the transparent component and is then conveyed by total internal reflection through an interface region between the wetting surface and the body fluid sample wetting the wetting surface to the detector.

14. A lancet, comprising:
a lancet body having a lancet tip for creating a puncture wound and a test area for optical examination of a body fluid;
a slot formed through the lancet body and extending from the tip into the test area, the slot defining a capillary to convey body fluid from the tip to the test area; and
a transparent component positioned in the test area and having a wetting surface for wetting a body fluid sample to be examined.

15. The lancet of claim 14, wherein the slot comprises a widened portion in the test area.

16. The lancet of claim 14, wherein the transparent component comprises a plastic sheet.

17. The lancet of claim 14, wherein the transparent component has a front side comprising the wetting surface, a first lateral face for coupling incoming light, and a second lateral face for decoupling outgoing light.

18. The lancet of claim 14, wherein the first and second lateral faces are positioned obliquely relative to the front side.

19. The lancet of claim 14, wherein the lancet body is substantially flat.

20. The lancet of claim 14, further comprising a pair of plastic sheets covering the test area.
